(19) United States
(12) Patent Application Publication
(10) Pub. No.: US 2003/0049236 A1
(43) Pub. Date: Mar. 13, 2003

(54) Immortalized stem cells

(75) Inventors: Moustapha Kassem, Lystrup (DK); Thomas G. Jensen, Skjodstrup (DK); Suresh I. S. Rattan, Arhus N. (DK)

Correspondence Address: BANNER & WITCOFF, 1001 G STREET N W, SUITE 1100, WASHINGTON, DC, 20001, US

(73) Assignee: Arhus Amt, Hojberg (DK)

(21) Appl. No.: 10/205,629

(22) Filed: Jul. 26, 2002

Related U.S. Application Data
(60) Provisional application No. 60/315,939, filed on Aug. 29, 2001.

(30) Foreign Application Priority Data
Jul. 27, 2001 (DK) PA 2001 01148

(51) Int. Cl. A61K 48/00; C12N 5/08; C12N 15/867

(52) U.S. Cl. 424/93.21; 435/456; 435/372

(57) ABSTRACT

The present invention discloses a method of immortalizing human stem cells by culturing human bone marrow stromal cells (hMSC), and transducting the cell cultures with a retroviral vector, comprising the human telomeric repeat subunit (hTRT) gene. Further, an immortalized stem cell line and its use are described. The immortalized stem cells may for example be used in the treatment of bone-fractures, bone loss associated with ageing and/or osteoporosis, and in tissue engineering.

### TECHNICAL FIELD OF THE INVENTION

The present invention relates to the field of regeneration and functional restoration of damaged and diseased tissue and to the manufacture of immortalized stem cell lines.

### BACKGROUND OF THE INVENTION

Human bone marrow contains a population of non-hematopoietic cells. These cells known as marrow stromal cells (MSC) play a role in supporting the hematopoietic cells (hence the name stroma). However, it has been increasingly recognised that these cells include a population of stem cells that are able to differentiate into several mesenchymal cell types including: osteoblasts, chondrocytes, adipocytes, and fibroblasts in addition to the marrow-supporting stroma. U.S. Pat. No. 5,942,225 describes lineage-directed induction of human mesenchymal stem cell differentiation. The molecular mechanisms controlling the differentiation of MSC into lineage-specific phenotype is not completely known. Detailed molecular studies on the cellular differentiation of MSC have been obstructed by the inability to obtain normal cells in a number large enough to allow such studies.

Mesenchymal stem cells (MSC), originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and possibly endothelium. Mesoderm also differentiates to visceral mesoderm, which can give rise to cardiac muscle, smooth muscle, or blood islands consisting of endothelium and hematopoietic progenitor cells. Primitive mesodermal or mesenchymal stem cells, therefore, could provide a source for a number of cell and tissue types. A tissue specific cell that has been named a stem cell is the mesenchymal stem cell, initially described by Fridenshtein, 1992.

Previous investigators have tried to overcome these limitations by creating immortalized marrow stromal cell lines and the large T-antigen (T) gene has been traditionally employed as an immortalizing agent (Hicok et al, 1998). Immortalization using the large T-antigen gene is known to induced several changes in the normal cell cycle machinery that may affect cells differentiation capacity and thus findings from T⁺-cell lines will be of limited value to understand the normal human physiology.

Telomeres are repetitive DNA sequences at the end of chromosomes that protect the ends of the chromosomes against degradation and prevent ligation of the ends of DNA by DNA-repair enzymes. The limited ability of normal cells for extensive proliferation has been suggested to be due the successive shortening of the telomeres during cell replication. A strong evidence supporting this hypothesis is the recent demonstration of an extended life span of human fibroblasts by forced expression of the human telomeric subunit gene (TRT) (Bodnar et al., 1998). The extended life span was not associated with evidence of transformation or tumor-genesis. Also, these cells maintain a pattern of onco-gene and tumor suppressor gene expression similar to normal cells.

In the present invention the inventors have employed a retroviral vector containing the human TRT gene to create an immortalized human marrow stromal cell line. It is demonstrated that MSCs overexpressing the human TRT gene exhibit an extended life span in culture and are able to differentiate into multiple lineages (e.g. osteoblasts, adipocytes and chondrocytes).

### SUMMARY

The present invention relates to a method of immortalizing human stem cells, comprising the following steps,

a) culturing human bone marrow stromal cells (hMSC),

b) transducting said cell cultures with a retroviral vector, comprising the human telomeric repeat subunit (hTRT) gene, and

c) obtaining an immortal stem cell line, wherein said immortalized cell line has substantially identical characteristics and properties when compared to the bone marrow stromal cells of step a).

The invention also concerns the use of the method. Furthermore, the invention discloses an immortalized stem cell line obtainable by the method of the invention and the use of said cell line.

### FIGURES

FIG. 1: shows the growth curve of immortalized cells compared to control cells. 980 controls and 980 TRT cells were grown in culture and their proliferative potential was measured as population doublings (PD).

FIG. 2: shows a telomerase assay for TERT cells.

FIG. 3: shows the DNA-sequence of the TERT coding fragment, which was incorporated into a retroviral vector. The fragment is from position 4215 to position 7668. Insert info: 4215 EcoR1 site, 4225 start of hTRT ORF (ATG codon), 7620 end of hTRT ORF, 7621 termination codon (TGA), 7626 end of hTRT cDNA sequence, 7668 EcoR1 site.

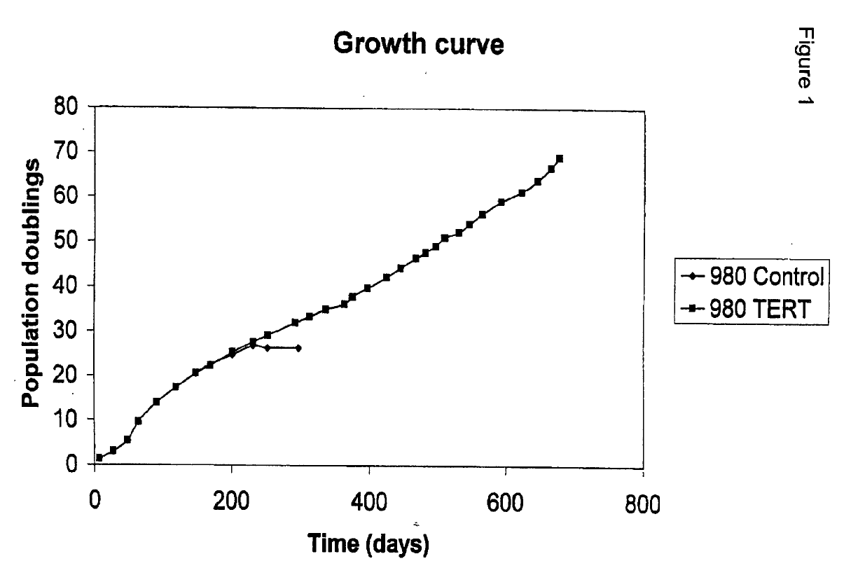

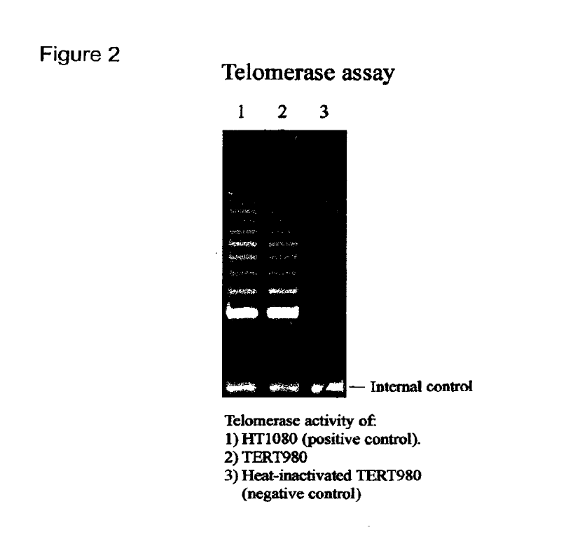

### DETAILED DESCRIPTION OF THE INVENTION

Accordingly, it is the purpose of the present invention to establish a pluripotential marrow stromal cell line by retroviral transfection with the human telomeric repeat subunit (TRT). The inventors have found a method of immortalizing human stem cells, comprising the following steps,

a) culturing human bone marrow stromal cells (hMSC),

b) transducting said cell cultures with a retroviral vector, comprising the human telomeric repeat subunit (hTRT) gene, and

c) obtaining an immortal stem cell line, wherein said immortalized cell line has substantially identical characteristics and properties when compared to the bone marrow stromal cells of step a).

Step a)

The human bone marrow stromal cells are cultured in accordance with standard conventional cell culturing methods known to the skilled person. An example of the method is described in the section “Examples” of the present specification.

Step b)

The hMSC cell cultures of the invention are transducted with a retroviral vector. The transduction method is preferably performed by the following steps:

b1) culturing a packaging cell line,

b2) constructing a retroviral vector,

b3) transfecting said packaging cell line, with said retroviral vector,

b4) transducing said packaging cell line with said transfected cells,

b5) transducing human bone marrow stromal cells with the packaging cells of step b4), said cells comprising said retroviral vector.

In the present context the definition of the term “transduction” is intended to cover the transfer of bacterial genes (DNA) from one bacterium to another by means of a bacterial virus (bacteriophage). There exist two kinds of transduction, namely specialised and general. In the case of specialised transduction, a restricted group of host genes becomes integrated into the virus genome. These “guest” genes usually replace some of the virus genes and are subsequently transferred to a second bacterium. In the case of generalised transduction, host genes become a part of the mature virus particle in place of, or in addition to the virus DNA. However, in the latter case the genes can come from virtually any portion of the host genome and this material does not become directly integrated into the virus genome.

Culturing Packaging Cells (b1)

The packaging cells are cells in which the retroviral vector of the invention is produced. In the present context it means a cell line capable of producing a vector which can transduce human cells. In the present invention the packaging cell line may be cells, such as PA317 or Phenix.

In a preferred embodiment the packaging cell line may be PG13 cells. This particular cell line is made in mouse embryo fibroblasts by transfection with the Gibbon Ape leukemia virus (GALV) env gene and the Moloney murine leukemia virus gag and pol genes. The PG13 cells are commercially available and may be obtained from ATCC, Rockville, Md.

According to the invention the PG13 cells may be cultured for 5-15 days, such as 7-13 days, for example 9-11 days before they are used to produce the retroviral vector.

Constructing a Retroviral Vector (b2)

In the present context the term “vector” is meant as an agent used to carry new genes into cells. Vectors may be plasmids, viruses or bacteria.

A “retrovirus” in the present context is used to carry new genes into cells. These molecules become part of that cells genome.

Retroviruses are divided into three subfamilies based primarily on their association with disease: oncovirinae, lentivirinae and spumavirinae. The retroviruses most widely used for gene transfer is Moloney murine leukemia virus which belong to the oncovirinae group.

Many versions of vectors based on Moloney murine leukemia virus have been developed. A vector termed MFG, mimics the normal pattern of mRNA splicing in the wildtype virus. This vector leads to high titer, efficient gene transfer and high expression levels.

The retroviral vector of the present invention comprises at least a fragment of the catalytic subunit of the human telomeric repeat (hTRT) gene or a variant thereof. A retrovirus (from the Latin word retrovir, which means “backward man”) is an oncogenic, single-stranded, diploid RNA (ribonucleic acid) virus containing (+) RNA in its virion and propagate through a double-helical DNA intermediate. It is called a retrovirus because its genetic information flows from RNA to DNA (reverse of normal). That is the virus contains an enzyme that allows the production of DNA using RNA as a template. Retroviruses can only infect cells in which DNA is replicating.

In a preferred embodiment of the invention the telomerase gene inserted into the retroviral vector (GCsam vector) is a hTERT cDNA fragment. Particularly, a 3452 base pair EcoR1 fragment from pGRN145 (Geron), or a variant thereof comprising all the coding sequences may be employed. According to the invention the important features of said vector are the efficiency of gene transfer, the ability to become integrated in the genome and the possibility for long term gene expression.

For the purpose of the invention retroviral vectors are best suited for the purpose of TERT-immortalization of stromal cells due to the efficient integration. However, it is also envisioned that the use of other vectors based on the Moloney murine leukemia virus or for example lentiviral vectors is within the scope of the present invention.

In yet another preferred embodiment the retroviral vector is driven by a Moloney murine leukemia virus long terminal repeat (GCSam).

Variant

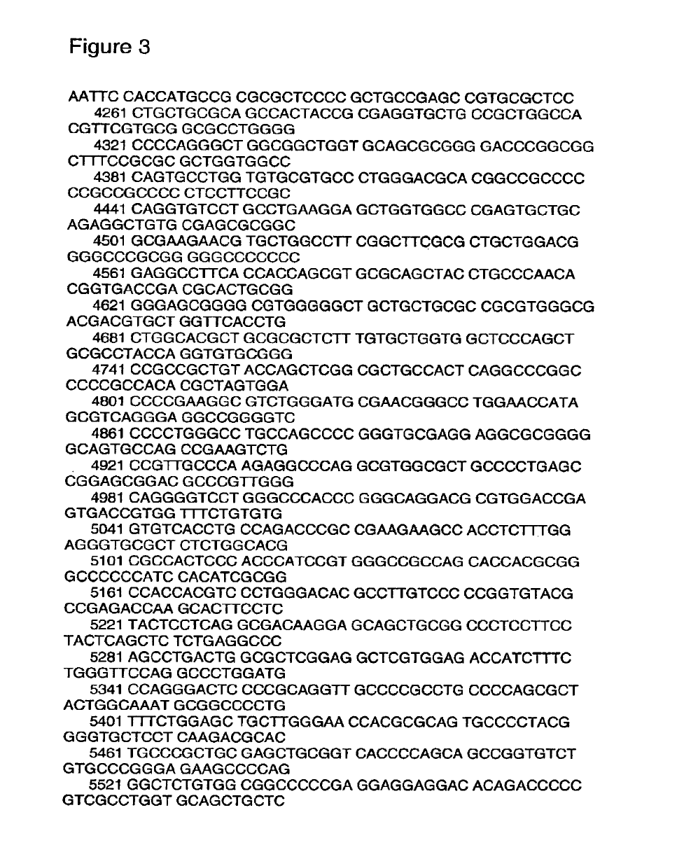

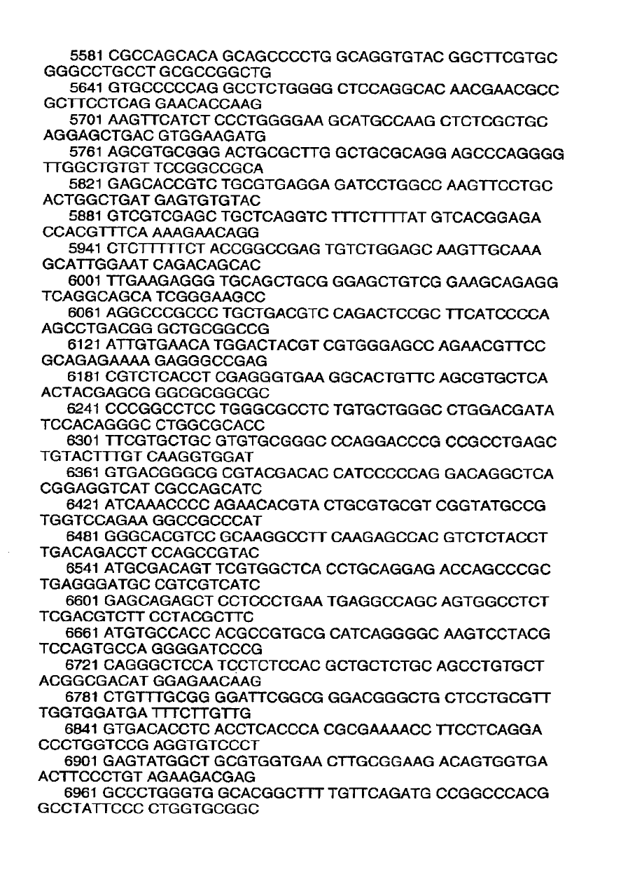

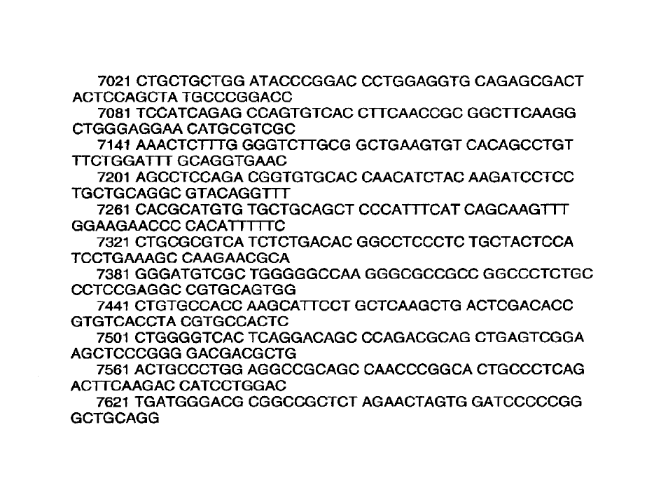

In the present context a variant means a functional equivalent of telomerase as identified by the sequence in FIG. 3 (3452 amino acids corresponding to the TERT protein), and functional equivalents of a fragment of telomerase comprising a predetermined amino acid sequence are defined as:

i) fragments comprising an amino acid sequence capable of being recognised by an antibody also capable of recognising the predetermined amino acid sequence, and/or

ii) fragments comprising an amino acid sequence capable of binding to a receptor moiety also capable of binding the predetermined amino acid sequence, and/or

iii) fragments having at least a substantially similar catalytic effect as the fragment of telomerase comprising said predetermined amino acid sequence.

The fragment of telomerase according to the present invention, including any variants and functional equivalents thereof, may in one embodiment comprise less than 5000 amino acid residues, such as less than 4500 amino acid residues, for example less than 4000 amino acid residues, such as less than 3500 amino acid residues, for example less than 3000 amino acid residues, such as less than 2500 amino acid residues, for example less than 2000 amino acid residues. Any functional equivalent may be obtained by addition, substitution or deletion of at least one amino acid.

For example certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity. In making such changes, the hydropathic index of amino acids may be considered. The importance of the hydropathic amino acid index in conferring interactive biologic function on a protein is generally understood in the art.

Transfection of Vector (b3)

In the present context the term “transfection” refers to a special case of transformation in which an appropriate recipient strain of bacteria is exposed to (free) DNA isolated from a transducing phage with the “take-up” of that DNA by some of the bacteria and consequent production and release of complete virus particles. The process involves the direct transfer of genetic material from donor to recipient.

The retroviral vector of the invention may be transfected into GGP+E86 packaging cell lines. These cell lines are made in NIH3T3 mouse fibroblasts by transfection with the gag, pol and env genes (Markovitz et al., 1988). Cells are obtained from ATCC, Rockville, Md.

Transducing Packaging Cell with Transfected Cells (b4)

The transfected cells GGP+E86 cells containing the retroviral vector are hereafter used to transduce the PG13 packaging cells.

Transducing Human Bone Marrow Stromal Cells with the Packaging Cells of Step b4), wherein the Packaging Cells Comprise a Retroviral Vector According to the Invention

In a further aspect of the invention the transduction of the human bone marrow stromal cells is at a time of division of said cells. It is preferred that the transduction is performed when the human bone marrow stromal cells have a density of 5-25%, preferably 10-20%. In the present context “density” is measured as the number of cells per predefined surface area of culture plates. The method of counting the cells in a counting chamber is known to the person skilled in the art.

According to the invention the transduction of the human bone marrow stromal cells is performed by using centrifugation. The centrifugation may be performed in the range between 200-2000 g, such as between 300-1800 g, for example between 500-1600 g, such as between 600-1400 g, for example between 700-1200 g, such as between 900-1100 g. The temperature during centrifugation may be between 25-37° C., such as between 27-35° C., for example between 29-33° C. In an aspect of the invention the centrifugation is performed for a period of between 5-55 minutes, such as between 10-50 minutes, for example between 15-45 minutes, such as between 20-40 minutes, for example between 25-35 minutes.

In one embodiment of the invention the centrifugation is performed at 1000×g and 32° C. for 30 minutes. It is speculated that the centrifugation step is crucial to obtaining the immortal stem cell line. The present inventors have found that the centrifugation step generally increases transduction ten-fold, such as nine-fold, for example eight-fold, such as seven-fold, for example six-fold, such as five-fold, for example four-fold, such as three-fold, for example two-fold. According to the invention the probability of obtaining immortalization is directly correlated to the transduction frequency.

Step C

It is an object of the present invention to obtain an immortal stem cell line, wherein the immortalized cell line has substantially identical characteristics and properties when compared to the bone marrow stromal cells of step a), i.e. bone marrow stromal cell which have not been immortalized.

By “substantially identical” is meant in the case where the inserted polynucleotide sequence is transcribed and translated to produce a functional polypeptide, one of skill will recognize that because of codon degeneracy a number of polynucleotide sequences will encode the same polypeptide. These variants include those sequences substantially identical (determined as described below) to a telomerase sequence encoding polypeptides that are either mutants of wild type telomerase polypeptides or retain the function of the telomerase polypeptide (e.g., resulting from conservative substitutions of amino acids in the telomerase polypeptide). In addition, variants can be those that encode dominant negative mutants as described below. Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or sub-sequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution.

The phrase “substantially identical” in the context of two nucleic acids or polypeptides, refers to sequences or sub-sequences that have at least 60%, preferably 80%, most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or sub-sequence complementarity when the test sequence has substantial identity to a reference sequence.

For sequence comparison, typically one sequence acts as a reference sequence, to which test sequences are compared. When using a sequence comparison algorithm, test and reference sequences are entered into a computer, sub-sequence co-ordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

The identity may be assessed by characterising and comparing the cell expression of certain cell markers, such as collagen type I or Alkaline phosphatase or any other relevant cell marker. The present invention overcomes the prior art problem of having immortalized cells differing molecularly and possibly also phenotypically from the initial cells used for immortalisation. This is an advantage when employing the immortalized stem cells of the present invention, for example for medical purposes, since it is important to apply immortalized cells identical to the originals to achieve successful applications.

Differentiation

It is within the scope of the invention that the human bone marrow stromal cells are capable of differentiating into cell types selected from, but not limited to the groups consisting of osteoblastic, adipocytic or chondrocytic phenotype.

Cell Line

In one embodiment of the invention the invention concerns an immortalized stem cell line obtainable by the method as described herein above. The immortalized stem cell line is characterised in that it comprises a hTRT (human telomeric repeat gene) vector.

Division

It is important for the success of the present invention that the time for transducing the human bone marrow stromal cells is precise. It is therefore of importance that the transduction of the human bone marrow stromal cells is performed at a time of division of the stromal cells. After transduction the stromal cells are cultured and later selected for immortalized cells. The cells which survive the prolonged culture period of for example several months are the ones comprising the vector of the invention. Therefore, in a preferred embodiment of the invention each cell of the transducted stromal cell line comprises a vector of the invention.

In a further aspect the transduction is performed when the human bone marrow stromal cells have a density of 5-25%, preferably 10-20% in culture.

In the present context a cell is defined as immortal when it has exceeded the double life span of the normal cell (i.e. non-immortalized cell). The maximal life span of a normal diploid cell in vitro varies dependent on the cell type (fetal versus adult cell) and culture conditions. Thus, the maximum life span of cultured normal cells in vitro is approximately 60-80 population doublings. For example keratinocytes may divide around 80 times, fibroblasts more than 50 times, and lymphocytes about 20 times. Normal bone marrowstromal cells may exhibit a maximal life span of 30-40 population doublings. According to the invention in one embodiment a stromal cell line may continuously grow past 350 population doublings and may still maintain a normal growth rate characteristic of young cells.

A crucial aspect of the present invention is that the transduction of the human bone marrow stromal cells is performed using centrifugation as described above.

Use

The present invention further relates to the use of the method for immortalizing stem cells as well as the use for an immortalized stem cell line according to the invention.

The present invention provides a method for using specifically differentiated cells for therapy, comprising administering the specifically differentiated cells to a patient in need thereof. It further provides for the use of genetically engineered pluripotent stem cells to selectively express an endogenous gene or a transgene, and for the use of MSCs grown in vivo for transplantation/administration into an animal to treat a disease. For example, neuroretinal cells derived from pluripotent stem or MSCs can be used to treat blindness, caused by among other things but not limited to neuroretinal disease, caused by among other things macular degeneration, diabetic retinopathy, glaucoma, retinitis pigmentosa.

The cells of the invention can be used to engraft a cell into a mammal, comprising administering autologous, allogenic or xenogenic cells, to restore or correct tissue specific metabolic, enzymatic, coagulation, structural or other functions to the mammal. The present cells can be used to engraft a cell into a mammal, causing the differentiation in vivo of cell types, and for administering the differentiated stem cells into the mammal.

In another embodiment the present cells, or their in vitro or in vivo differentiated progeny, may be used to correct diseases, such as a genetic disease, degenerative disease, cardiovascular disease, metabolic storage disease, neural disease and/or a cancer disease. In another embodiment the cells may be used to produce gingiva-like material for treatment of periodontal disease and in a further embodiment the present cells may be to be used to develop skin epithelial tissue derived from pluripotent stem cells utilized for skin grafting and plastic surgery. They may be used to produce blood ex-vivo for therapeutic use, or to produce human hematopoietic cells and/or blood in prenatal or postnatal animals for human use.

Further in one aspect of the invention the cells may be used as a therapeutic to aid for example in the recovery of a patient from chemotherapy or radiation therapy due to the treatment of cancer, or be used in the treatment of auto-immune disease, or the cells may be used to induce tolerance in the recipient, such as in the treatment of AIDS or other infectious diseases.

According to the invention the MSC differentiated into cardiomyocytes may be used to treat cardiac diseases including among others but not limited to myocarditis, cardiomyopathy, myocardial infarction, hypertension, atherosclorosis, heart valve dysfunction. A genetically engineered pluriipotent mammalian derived stem cell, or its differentiated progeny, may be used to treat a disease with Central Nervous System (CNS) deficits or damage. Further the present pluri potent mammalian derived stem cell, or its neuronally related differentiated cell, may be used to treat a disease with neural deficits or degeneration including among but not limited to stroke, Alzheimer's, Parkinson's disease, Huntington's disease, AIDS associated dementia, spinal cord injury, metabolic diseases effecting the brain or other nerves.

It is an object of the invention to provide a pluripotent mammalian derived stem cell, or their differentiated progeny such as stromal cells may be used to support the growth and differentiation of other cell types in vivo or in vitro, including but not limited to hematopoietic cells, pancreatic islet insulin-producing β cells, and hepatocytes.

The present stem cell, or cartilage differentiated progeny, may be used to treat a disease of the joints or cartilage including but not limited to cartilage tears, cartilage thinning, osteoarthritis. Moreover, the stem cells of the invention, or their osteoblast differentiated progeny may be used to ameliorate a process having deleterious effects on bone including among but not limited to bone fractures and non-healing fractures. “defects” in bones caused by the removal of primary bone tumors, or bone metastases spreading to said bones, such as in breast cancer or lung cancer.

Bone Loss

One such purpose may be for the treatment of bone loss associated with ageing and/or osteoporosis. The immortalized cells will be able to replace cells lost during such diseases.

Bone Fracture

Another application of the invention is for use in the treatment of bone-fractures.

Prosthesis Coating

In the field of orthopaedic surgery, prosthesis loosing with age is a major problem for joint replacement technology. It is envisaged that coating the prosthesis surfaces with the immortalized cell line of the invention loaded on a carrier can increase the adhesion of the prosthesis to the bone and prevent loosening.

Endocrine Replacement Therapy

In another embodiment of the invention the cells are ideal candidates for deriving endocrine-hormone secreting cells (e.g. insulin-producing, growth hormone-producing) from the present cell line. This may be done either through induction of differentiation of stem cells into these cell types by chaning culture conditions or through transfection of the cells with genes responsible for hormone synthesis and secretion. Such modified cells may be used for treatment of hormone deficiency, for example diabetes mellitus or growth hormonedeficiency.

Tissue Engineering

In yet a further aspect the immortalized stem cell line may be used for tissue engineering, such as for creating bone matrix for the treatment of bone defects and non-healed fractures and for creating cartilage matrix for the treatment of cartilage defects and osteoarthritis. In general the stem cells may be used for creating general artificial tissues in vitro, such as skin, cartilage, heart valves and vascular tissues, all which can be used in replacement therapy.

It is also within the scope of the invention to use the immortalized cells to create a skin matrix for the treatment of skin defects (including cosmetic purposes) after skin burns.

Growth Factor Production

The immortalized stem cells of the invention may be used in the production and secretion of growth factors, such as VEGF (Vascular Endothelial Growth Factor), PDGF (Platelet Derived Growth Factor), Insulin-like growth factors (IGFs) and others.

Vehicle for Gene Therapy “Metabolic Sink”

In another aspect of the invention the immortalized stem cells are used as “metabolic sink”. For example it is envisioned that the immortalized stem cells of the present invention are capable of metabolising substances accumulating during the course of various diseases. Such metabolites could be Phenylalanine in phenylketonuri (PKU); Ornithine in Gyrate Atrophy or Adenosine in Adenosine deaminase (ADA) deficiency, cholesterol in hypercholestremic states.

Within the application area of the “metabolic sink” the immortalized cells may produce metabolic enzymes, such as Adenosine Deaminase (ADA), Ornithin Aminotransferase (OAT) or Phenylalanine Hydroxylase (PAH).

Vehicle for Drug Delivery

According to the invention the use of the immortalized stem cells may be for drug delivery of compounds, such as Coagulation factor VIII (Hemophilia A), Coagulation factor IX (Hemophilia B), Erythropoietin (EPO), insulin, leptin, angiostatins/endostatins, human growth hormone and/or interleukins (II).

The main routes of drug delivery, in the treatment methods are intravenous, oral, and topical. Other drug-administration methods, such as subcutaneous injection or via inhalation, which are effective to deliver the drug to a target site or to introduce the drug into the blood stream, are also contemplated.

In one embodiment of the invention the administration is systemic. The cells are intravenously injected, whereby the cells are positioned with for example bone marrow or bone structures.

In a further embodiment of the invention the administration is local. Immortalized stem cells may be implanted subcutanously, such as in immunoprotective capsules or in bone structures.

Drug Testing

In yet another aspect of the invention the immortalized stem cells are used for drug testing, such as for identifying drugs acting on mesenchymal stem cells and/or drugs enhancing osteoblast differentiation, chondrocyte differentiation, neuronal cell differentiation and/or muscle cell differentiation,. This provides for a unique opportunity of testing drugs on normal human cells capable of differentiation in a multitude of cell types. The present invention represents a good alternative for drug testing on small animals, and is beneficial in drug discovery studies.

Gene Therapy

According to the invention the immortalized stem cells may be employed in gene therapy.

Genetic material, such as genes or functional parts thereof may be introduced in constructs as a single DNA molecule encoding all of the genes, or different DNA molecules having one or more genes. The constructs may be introduced simultaneously or consecutively, each with the same or different markers.

The gene may be linked to the complex as such or protected by any suitable system normally used for transfection, such as viral vectors of the invention or artificial viral envelope, liposomes or micellas, wherein the system is linked to the complex.

Numerous techniques for introducing DNA into eukaryotic cells are known to the person skilled in the art. Often this is done by means of vectors, and often in the form of nucleic acid encapsidated by a (frequently virus-like) proteinaceous coat. Gene delivery systems may be applied to a wide range of clinical as well as experimental applications.

Vectors containing useful elements such as selectable and/or amplifiable markers, promoter/enhancer elements for expression in mammalian, particularly human, cells, and which may be used to prepare stocks of construct DNAs and for carrying out transfections are well known in the art and are also within the scope of the invention. Many are commercially available.

Various techniques have been developed for modification of target tissue and cells in vivo. A number of virus vectors are known which allow transfection and random integration of the virus into the host. Routes and modes of administering the vector include injection, e.g intravascularly or intramuscularly, inhalation, or other parenteral administration.

Protein Production

In a further embodiment of the invention the immortalized cells are used for large scale production of pure proteins which may then be used in crystallographic characterization.

### EXAMPLES

The following are examples of non-limiting embodiments of the invention.

Cell Source and Cell Separation Procedure

After approval by The Regional Scientific-Ethical Committee for Aarhus County, and a signed consent from each donor, human bone marrow stromal cells (hMSc) were obtain by aspiration of 10-15 ml bone marrow from the iliac crest of healthy subjects. hMSc were prepared as previously described (Rickard D. J. et al. 1996 and Kassem M. et al. 1991). Briefly, the samples were diluted in cell culture medium and centrifuged at 1500 g for 25 min. on a Lymphoprep® cushion (Nycomed DAK, Denmark). The low density mononuclear cells were collected and seeded at a concentration of 130.000 (hMSc) cells per cm². Cells were incubated at 37° C. in 5% CO₂, and the medium was replaced once a week. The non-adherent cell populations also present in the mononuclear fraction were removed by washing at each medium changing.

Medium and Reagents

Modified essential medium (MEM) (Life Technology, Denmark) containing 10% Foetal Calf Serum (FCS) (Bio-Whittaker, Belgium) was used for all cell types. MEM containing 15% Normal Horse Serum (Sigma, USA) and 10⁻⁷M Dexamethasone (Sigma, USA), and MEM containing 290 nM Ascorbic Acid, were used respectively to induce adipocytic differentiation and mineralisation. 1.25-vitamin D₃ (vit. D) (Hoffman La Roche, Denmark) treatment was carried out for 48 hrs with a concentration of 10⁻⁸M in standard medium.

Telomerase Immortalisation of Human Bone Marrow Cells

Transduction of the telomerase gene into hMSc.

The telomerase gene and the green fluorescence protein (GFP) gene (as control) inserted in the GSsam retroviral vector (from T. G. Jensen, Aarhus, Denmark) were used to immortalise hMSc. These retroviral vectors are driven by a Moloney murine leukemia virus (MoMLV) long terminal repeat (LTR), and they were packaged in the gibbon ape leukemia virus (GALV) PG13 packaging cell line. Supernatants from the PG13 virus producing cells were collected and used to transduced hMSc. 9 days old in culture hMSc plated on a 6 well/plate were exposed to the virus supernatants containing 8 μg/ml polybrene. The plates were centrifuged for 30 min. at 1000×g at 32° C., and then incubated for 2 hrs. at 37° C. Medium was then changed and cells were monitored for GFP expression during the next two days, in order to asses the quality of transduction. Cell medium was changed once a week and the cells were passaged at confluence.

Cell culture. Packaging cell lines (ATCC, Manassas, Va., USA) were cultivated in high glucose (4.5 g/l) Dulbecco's modified Eagle's medium (DMEM, Gibco, Life Technology, Rockville, Md., USA) supplemented with 10% fetal bovine serum (Gibco, Life Technology), 100 U/ml of penicillin and streptomycin (Gibco, Life Technology) and 2 mM of L-glutamine (Gibco, Life Technology).

Retroviral vectors and packaging cell lines. The vector pGRN1212 containing the human TERT gene (hTERT) was kindly provided by Geron Corp., Ca., USA (see FIG. 3). The retroviral vector GCsamTERT which does not contain a neo gene was constructed by inserting the hTERT gene, isolated from pGRN1212, into the retroviral vector GCsam (Chuah et al, 1995). In this vector the expression of the transgene is driven from a Moloney Murine Leukemia Virus LTR. GCsamTERT was packaged in PG13 as described (Onodera et al, 1997). GCsamTERT DNA was transfected into GP+E86 (Markovitz et al., 1988) by calcium phosphate co-precipitation (Mammalian transfection kit, Stratagene, La Jolla, Calif.). Supernatant from transfected cells was harvested 16 hours later, supplemented with polybrene to a concentration of 8 μg/ml, passed through a 0.45 μm filter, and used to transduce PG13 cells by using a centrifugation technique (Bunnell et al, 1995). A population of the packaging cell line was used for production of the retroviral vector. The retroviral vector GCsamGFP, containing the eGFP gene, was constructed and packaged as described previously (Jensen et al, 2000).

Transduction of stroma cells. Transduction of stroma cells was performed using a centrifugation technique (Bunnell et al, 1995). Polybrene (8 μg/ml) was added to fresh supernatant from packaging cell lines cultivated at 37° C., and the supernatants were passed through a 0.45 μm filter. Stroma cells, seeded the day before at a confluency of 10-20%, in a well of a 6 well plate (NUNC) were treated with 2 ml of retroviral supernatant. Plates were centrifuged 30 minutes at 32° C. at 1000×g and then incubated at 37° C. 16 hours. Thereafter, the supernatants were removed, and cells were refed with fresh complete media.

Telomere Repeats Assay Protocol (TRAP)

The telomerase activity was assessed by TRAP using the TRAPeze Telomerase Detection Kit, Oncor, USA, based on the protocol first described by Kim et al. Briefly, 100000 cells were pelleted and lysated in 30 μl of a CHAPS containing lysis buffer. 2 μl of the lysate were incubated for 30 min. at 30° C. to promote amplification. The amplification products were then subjected to 2 step PCR and the PCR products were visualised with an Egle Eye System after PAGE and staining in Ethidium Bromide solution.

Cell Growth

Long-term growth. At confluence, the cells were passaged by trypsinisation at a split ratio of 1:4. The number of cells was manually counted in a Bürker-Turk hematocytometer, and the number of Population Doublings (PD) was calculated using the formula InN/In2 where N is the cell number of the confluent monolayer divided by the initial number of cell seeded.

Cells were considered to have reached the end of their lifespan when they failed to become confluent within 6 weeks of culturing.

Staining for Senescence-Associated β-galactosidase Activity

In order to monitor the emergence of senescent phenotype, hMSc were stained for β-galactosidase. Cells were washed in PBS, fixed in 2% formaldehyde/0.2% glutaraldehyde in PBS for 3-5 min at room temperature. The cells were then incubated at 37° C. with freshly made -galactosidase stain solution (1 mg 5-bromo-4-chloro-3-indolyl β-D-galactosidase [Sigma, USA] per ml, 40 mM Citric acid sodium phosphate, pH. 6.0, 5 mM Potassium ferrocyanide, 150 mM NaCl, and 2 mM MgCl₂). Staining reaction was stopped after 16 hrs. incubation.

RNA Extraction, cDNA Synthesis, and RT-PCR

RNA was extracted from cultures of confluent hMSc, by Trizol® (GIBCO, BRL, UK) following the manufacture protocol. RNA concentration was measured at 260 nm, and RNA was then stored at −20° C. First strand cDNA was synthesised from 4 μg of total RNA using 50 U/μl Mulv-reverse transcriptase (PE Biosystem, Denmark) in 15 μl reaction volume. Hexamer random primer (50 pmol/μl) and dNTPs (10 mM) were from Roche Molecular Biochemicals, Switzerland; 10× PCR buffer, 25 mM MgCl₂, 50 μM Oligo dT, 20 U/μl RNAse inhibitor were from PE Biosystem, Denmark. cDNA synthesis was run in a Perkin Elmer GeneAmp® PCR System 9700 at 23° C. for 5 min., 42° C. for 240 min., and 95° C. for 5 min. cDNA was stored at −20° C. until use.

PCR was run using Amplitaq gold, buffer, and reagents from PE Biosystem, Denmark, in a final volume of 20 μl per sample [10× PCR buffer, MgCl₂ 1.5 mM, dNTP 0.2 mM each dATP, dGTP, dCTP, and dTTP, and Amlitaq Gold polymerase (0.025 U/μl). Primers purchased from DNA Technology, Denmark (table 2) were used at a final concentration of 0.5 pmol/μl, except for GAPDH primers that were used at a concentration of 0.1 pmol/μl.

Alkaline Phosphatase Staining

Cells grown to 70-90% confluence were fixed in 37% formaldehyde/methanol in a 1:9 ratio. The alkaline phosphatase presence in the cell membrane was detected by exposing the cells to a propandiol solution containing 1-Naphthyl phosphate sodium salt (monohydrate) from Merck as substrate for the phosphatase and Variamine Blue B salt from Fluka Chemika as stain. A brown precipitate was detectable after 1 hr incubation at 4° C.

Adipocytic Differentiation

Cells grown in adipocytic medium for 14 days were fixed in 4% neutral buffer formalin for 10 min. followed by a wash with 3% isopropanol. Cells were then stained with an Oil red O solution (0.5 g Oil red O, 100 ml isopropanol, and 67 ml water) for 1 hr at room temperature.

After staining the cells were counterstained with Mayers Hematoxylin.

Mineralisation

Mineralisation was promoted by growing the cells for 14-22 days in medium containing Ascorbic acid. Calcium precipitates were detected after fixation in ice cold 70% ethanol for 1 hr, by staining with 40 mM Alizarin Red S pH. 4.2 in 10 min.

In vivo Bone Formation

Hydroxyapatite/tricalcium phosphate (HA/TCP) (Zimmer Scandinavia, Denmark) was transferred to a Nunc cryotube and wetted with 200 ul MEM containing 1% penicillin/streptomycin and 10% heat inactivated FCS. Human MSC trypsinized and incubated with HA/TCP granules for 30 min with rotation (20 rpm) and then incubated for 1 h at 37° C. HA/TCP loaded with cells was then transplanted subcutaneously inot NOD/LtSz-scid/scid mouse (6-week old females, ca. 20 g). Transplantation was performed under general anathesia achieved by intraperitoneal injection of ketamin (2.5 mg/ml) and xylazin (4 mg/ml) in phyisiological saline at 0.1 ml/g body weight. Mice were shaved and the skin was disinfected with 70% ethanol prior to operation. Two midlongitudinal skin incisions of about 1 cm were made on the dorsal surface of each mouse and subcutaneous pockets were formed by blunt dissection. A singel transplant was placed into each pocket. The incision were closed. After 8 weeks the transplants were recovered, fixed in ethanol (70%) and embedded undecalcified in methyl-methacrylate. Tissue sections were cut 7.5 um thick and were stained for Goldner-trichrome.

Expression of Human TERT Gene in hTERT⁺-MSC

Using TRAP to deterime the activity of TERT gene in the hTERT⁺-MSC. It was found that hTERT⁺-MSC were able to express telomerase activity and teomerase activity was maintained irrespective of cells' population doubling levels suggesting a stable expression of the human TERT gene. Telomerase activity was not detectable in different strains of normal human MSC including the parental strain of the TRT⁺-MSC. The integration site and copy number of the ectopic hTERT gene were determined using fluorescent in situ hybridization (FISH). Twenty metaphases and 100 nuclei were evaluated. The ectopic hTERT was found to be integrated at chromosome 5q23-31. All nuclei showed only one copy of ectopic hTERT and two copies of endogenous hTERT.

Extended Life-span of Human TRT⁺-MSC

Human normal non-transfected MSC had a limited life span in cultures with maximal population doubling levels of 30. The TRT⁺-MSC showed an extended life span and has currently undergone 350 population doublings and has been cultured continuously for more than 3 years. The cells do not show any morphological evidence of senescent phenotype.

Staining of TRT⁺-MSC for β-galactosidase Activity

As a marker of senescent phenotype, staining for β-galactosidase (β-gal) activity at neutral pH was employed. At the end of their life span, the senescent phenotype of normal MSC was associated with the positive staining for β-gal in more than 90% of the cells. Staining for the β-gal was performed regularly at different population doubling levels of the TRT⁺-MSC. Only a small and a stable proportion (less than 10%) of the cells stained positive for β-gal suggesting the maintenance of a “young” phenotype in spite of the extensive proliferation.

Expression of the Osteoblastic Phenotype by the TRT-cells

In order to investigate the cellular phenotype characteristics of TRT⁺-MSC, the ability of the cells to express known osteoblastic phenotype markers was examined. TRT+-MSC were found to express constitutively known osteoblast-specific genes: alkaline phosphatease (AP), osteopontin, core binding factor 1 (CBFA-1) and osteocalcin (OC) in presence of calcitriol (10⁻⁷M). The expression levels of these osteoblast-specific genes were found to be stable during the extended period of growth in culture. In addition to mRNA expression, MSCs stained positive for AP and type I collagen (Coll) confirming the observed findings on the mRNA level. Employing an assay for in vitro mineralization, the cells were able to form a mineralized matrix in vitro at confluency as demonstrated by staining by alizarin red.

Expression of the Adipocytic Phenotype of the Cells

As MSC are able to differentiate into adipocytes, it was examined whether TRT⁺-MSC maintained the adipocytic-forming capacity. TRT⁺-MSC were incubated with adipogenic medium containing normal horse serum and dexamethasone (10⁻⁷M). After incubation for 2 weeks, TRT⁺-MSC were able to form morphologically recognisable adipoctyes with oil-filled cytoplasm and showed positive histochemical staining for oil red O. Also, the cells expressed constitutively lipoprotein lipase and proxisome proliferator activator receptor-gamma (PPAR-γ) known adipocyte-specific genes.

In vitro Bone Formation Assay

The ultimate proof of the osteoblastic nature of the cells is their ability to form bone. In order to examine the ability of the hTERT⁺-MSC to form bone, the cells were transplanted subcutaneously in immune-deficient mouse. hTERT⁺-MSC were able to form a trabecular network that enclosed marrow cells and adipocytes. Immunocytochemical staining of implants using human specific antibodies, have demonstrated that bone matrix is of donor origin (human) while the hematopoietic cells were of recipient origin (mouse). Quantitative analysis of the amount of bone formed showed that hTERT-MSC formed a larger amount of bone compared to normal un-transduced cells.

Differentiation into Other Cell lineages

The differentiation potential of hTERT-MSC were tested by culturing the cells in different combinations of growth factors and cyokines to induce a lineage specific phenotype. The cells were able to express markers for neuronal cells, myocytic cells, endothelial cells and B-lymphocytes.

### References

Bodnar A. G., Cuellette, M., Frolkis, M. et al. (1998). Extension of life-span by introduction of telomerase into normal human cells. Science, 279: 349-352.

Bunnell, B. A, Muul, L. M, Donahue, R. E., Blaese, R. M., Morgan, R. A. (1995). High-efficiency retroviral-mediated gene transfer into human and nonhuman primate peripheral blood lymphocytes. Proc. Natl. Acad. Sci. USA 15:92(17):7739-43.

Chuah, M. K., Vandendriessche, T. & Morgan, R. A. (1995) Development and analysis of retroviral vectors expressing human factor VIII as a potential gene therapy for hemophilia A. Hum. Gene Ther. (6): 1363-1377.

Fridenshtein, Arkh. (1982). Patol., 44: 3-11,

Garlick J A, Katz A B, Fenjves E S, Taichman L B. Retrovirus-mediated transduction of cultured epidermal keratinocytes (1991). J Invest Dermatol;97:824-9.

Hicok, K., Thomas, T., Gori, F., Rickard, D. J., Spelsberg, T. C., Riggs, B. L., (1998). Development & characterisation of conditionally immortalized osteoblast precursor cell lines from human bone marrow stroma. J. Bone Miner. Res. 13: 205-217.

Jensen, U. B., Petersen, M. S., Lund, T. B., Jensen, T. G., Bolund, L., (2000) Transgene expression in human epidermal keratinocytes: cell cycle arrest of productively transfected cells. Exp. Dermatol. 9(4):298-310.

Markowitz, D., Goff, S. & Bank, A. (1988) A safe packaging line for gene transfer: separating viral genes on two different plasmids. J. Virol. 62, 1120-1124.

Miller, A. D., Garcia, J. V., von Suhr, N., Lynch, C. M., Wilson, C., & Eiden, M. V. (1991) Construction and properties of retrovirus packaging cells based on gibbon ape leukemia virus. J Virol. 65, 2220-4.

Onodera M, Yachie A, Nelson D M, Welchlin H, Morgan R A, Blaese R M (1997) A simple and reliable method for screening retroviral producer clones without selectable markers. Hum Gene Ther 1;8(10):1189-94.

1. A method of immortalising human stem cells, comprising the following steps,
a) culturing human bone marrow stromal cells (hMSC),
b) transducting said cell cultures with a retroviral vector, comprising the human telomeric repeat subunit (hTRT) gene, and
c) obtaining an immortal stem cell line, wherein said immortalized cell line has substantially identical characteristics and properties when compared to the bone marrow stromal cells of step a).

2. The method according to claim 1, wherein step b) comprises the following steps,
b1) culturing a packaging cell line,
b2) constructing a retroviral vector,
b3) transfecting into said packaging cell line, said retroviral vector
b4) transducing said packaging cell line with said transfected cells,
b5) transducing human bone marrow stromal cells with the packaging cells of step b4), said cells comprising said retroviral vector.

3. The method according to claim 2, wherein the retroviral vector is driven by a Moloney murine leukemia virus long terminal repeat (GCSam).

4. The method according to the claims 2-3, wherein the retroviral vector is produced in PG13 cells.

5. The method according to the claims 3-6, wherein the PG13 cells are cultured for 5-15 days.

6. The method according to any of the preceding claims, wherein the transduction of the human bone marrow stromal cells is at a time of division of said cells.

7. The method according to claim 6, wherein the transduction is performed when the human bone marrow stromal cells have a density of 5-25%, preferably 10-20%.

8. The method according to claim 2, wherein the transduction in step b5) is performed using centrifugation.

9. The method according to claim 8, wherein the centrifugation is performed at 1000×g and 32° C. for 30 minutes.

10. The method according to any of the preceding claims, wherein the humane bone marrow stromal cells are capable of differentiating into cells selected from the groups consisting of osteoblastic, adipocytic, chodrocytic or myocytic phenotypes.

11. An immortalized stem cell line obtainable by the method as defined in claim 1.

12. An immortalized stem cell line, characterised in that it comprises a vector as defined in claim 1.

13. The immortalized stem cell line according to any of the claims 11-12, wherein the human bone marrow stromal cells of step a) in claim 1 are cultured.

14. The immortalized stem cell line according to any of the claims 11-12, wherein step b) of claim 1 comprises the following steps,
d) culturing a packaging cell line,
e) constructing a retroviral vector,
f) said retroviral vector being transfected into GP+E86,
g) transducing said packaging cell line with said transfected cells,
h) transducing human bone marrow stromal cells with the packaging cells of step g), said cells comprising said retroviral vector.

15. The immortalized stem cell line according to any of the claims 11-12, wherein the retroviral vector is driven by a Moloney murine leukemia virus long terminal repeat (GCSam).

16. The immortalized stem cell line according to any of the claims 11-12 and the claims 13-15, wherein the retroviral vector is produced in PG13 cells.

17. The immortalized stem cell line according to claim 16, wherein the PG13 cells are selected from second or third generation cells.

18. The immortalized stem cell line according to claim to the claims 16-17, wherein the PG13 cells are cultured for 5-15 days.

19. The immortalized stem cell line according to any of the claims 11-12 and the claims 13-18, wherein the transduction of the human bone marrow stromal cells is at a time of division of said cells.

20. The immortalized stem cell line according to any of the claims 11-12 and the claims 16-22, wherein the transduction is performed when the human bone marrow stromal cells have a density of 5-25%, preferably 10-20% in culture.

21. The immortalized stem cell line according to claim 20, wherein the transduction in step g) is performed using centrifugation.

22. The immortalized stem cell line according to claim 21, wherein the centrifugation is performed at 1000×g and 32° C. for 30 minutes.

23. The immortalized stem cell line according to any of the claims 11-12 and the claims 13-22, wherein each cell of said cell line comprise a vector as defined in claim 1.

24. The immortalized stem cell line according to any of the claims 11-12 and the claims 13-23, wherein the humane bone marrow stromal cells are capable of differentiating into immortalized cells selected from the groups consisting of osteoblastic, adipocytic, chodrocytic or myocytic phenotypes.

25. Use of the method as defined in the claims 1-10.

26. Use of the immortalized stem cell line as defined in the claims 11-24.

27. The use according to claim 26 for the treatment of bone loss associated with ageing and/or osteoporosis.

28. The use according to claim 26 defined for the treatment of bone-fractures.

29. The use according to claim 26 for tissue engineering, such as for creating bone matrix for the treatment of bone defects and non-healed fractures.

30. The use according to claim 26 for tissue engineering, such as for creating cartilage matrix for the treatment of cartilage defects.

31. The use according to claim 26 for tissue engineering, such as for creating skin matrix for the treatment of skin defects and skin burns.

32. The use according to claim 26 for tissue engineering, such as for producing and secreting growth factors, such as VEGF, PDGF and hGH.

33. The use according to claim 26 for metabolic sink.

34. The use according to claim 33, wherein the metabolic sink comprises lowering the level of blood cholesterol.

35. The use according to claim 34, wherein the metabolic sink comprises immortalized cells as defined in the claims 10-24 producing metabolic sink enzymes, such as Adenosine Deaminase (ADA), Ornithin Aminotransferase (OAT) or Phenylalanine Hydroxylase (PAH).

36. The use according to claim 26 for drug delivery of compounds, such as Coagulation factor VIII Hemophilia A), Coagulation factor IX (Hemophilia B), Erythropoietin, insulin, leptin, angiostatins/endostatins, human growth hormone and/or interleukins (II).

37. The use according to claim 26 for drug testing, such as for identifying drugs acting on mesenchymal stem cells and/or drugs enhancing osteoblast differentiation and/or drugs enhancing chondrocyte differentiation.

38. The use according to claim 26 for gene therapy.

39. The use according to claim 26 for production of high amounts of pure protein for crystallisation.
